(19) United States
(12) Patent Application Publication
(10) Pub. No.: US 2015/0258006 A1
(43) Pub. Date: Sep. 17, 2015

(54) THREE-PHASE HAIR CONDITIONER

(71) Applicant: Henkel AG & Co. KGaA, Duesseldorf (DE)

(72) Inventors: Jens Delowsky, Norderstedt (DE); Thomas Hippe, Appen (DE); Sandra Fuchs, Pinneberg (DE)

(21) Appl. No.: 14/722,256

(22) Filed: May 27, 2015

Related U.S. Application Data
(63) Continuation of application No. PCT/EP2013/071993, filed on Oct. 21, 2013.

(30) Foreign Application Priority Data
Nov. 30, 2012 (DE) 10 2012 221 957.9

(51) Int. Cl. A61K 8/891; A61K 8/34; A61K 8/64; A61K 8/65; A61Q 5/12; A61K 8/92

(52) U.S. Cl. CPC A61K 8/891; A61Q 5/12; A61K 8/922; A61K 8/645; A61K 8/65; A61K 8/342; A61K 2800/5426

(57) ABSTRACT

An agent for the treatment of keratin fibres, in particular human hair, includes at least one selected dimethicone and/or dimethiconol and at least one selected oil in a cosmetic carrier including at least 80.0% by weight, in relation to the total weight of the agent, aqueous carrier.

### FIELD OF THE INVENTION

The present invention generally relates to an agent for treating keratin-containing fibers, in particular human hair, which includes at least one selected dimethicone and/or dimethiconol and at least one selected oil in a cosmetic carrier with at least 80.0 wt. % of an aqueous carrier, based on the total weight of the agent.

### BACKGROUND OF THE INVENTION

Care products for keratinic fibers often have the disadvantage that they weigh down the hair and thus reduce its fullness. This problem occurs in particular with products that are left on the hair (so-called leave-on products). In contrast, products that are rinsed out shortly after being applied (so-called rinse-off products) often do not have sufficient care potential. It is therefore desirable to provide hair treatment agents that provide lasting care for the hair and improve the combability, gloss and handle without weighing down the style. Ideally, it should be possible to achieve this both with products that are rinsed off the hair again within a relatively short time after being applied and with those that can remain on the hair.

It is also desirable to improve the stability of such compositions. Conventional prior two-phase care products exhibit marked instability under mechanical load. These loads can be, for example, shaking prior to application or delivery from a spray head, since high shear forces occur during these operations. At low temperatures too, however, the conventional compositions are insufficiently stable, particularly if a high mechanical load additionally occurs at low temperatures. Deep hair conditioners as three-phase compositions are consequently much more difficult to develop. Up to the present, therefore, no three-phase deep conditioners are either available on the market or described in the specialist literature.

Furthermore, other desirable features and characteristics of the present invention will become apparent from the subsequent detailed description of the invention and the appended claims, taken in conjunction with the accompanying drawings and this background of the invention.

### BRIEF SUMMARY OF THE INVENTION

Unpredictably, it has now been found that haircare agents which include an aqueous carrier can be improved significantly in terms of their care potential and effect on fullness and volume as well as their stability under high mechanical loads and at low temperatures if they meet certain requirements in relation to the ingredients and the quantities thereof.

According to the invention, a hair treatment agent includes, based on its weight, at least 80.0 wt. % of an aqueous carrier, at least 0.1 to 15.0 wt. % of at least one dimethicone and/or dimethiconol with a viscosity of 100 to 350 cSt, measured by the Dow Corning test method CTM 0004, and at least 0.1 to 15.0 wt. % of at least one oil. The agent is furthermore characterized in that the composition includes three phases that are visibly separate from one another.

### DETAILED DESCRIPTION OF THE INVENTION

The following detailed description of the invention is merely exemplary in nature and is not intended to limit the invention or the application and uses of the invention. Furthermore, there is no intention to be bound by any theory presented in the preceding background of the invention or the following detailed description of the invention.

The present invention provides hair treatment agents that include, based on their weight,
a) at least 80.0 wt. % of an aqueous carrier,
b) at least 0.1 to 15.0 wt. % of at least one dimethicone and/or dimethiconol with a viscosity of 100 to 350 cSt, measured by the Dow Corning test method CTM 0004, and
c) at least 0.1 to 15.0 wt. % of at least one oil and
d) furthermore characterized in that the composition includes three phases that are visibly separate from one another.

The agents according to the invention include at least 80.0 wt. % of an aqueous carrier. Preferred agents according to the invention are characterized in that they include—based on their weight—85.0 to 98.0 wt. %, preferably 80.0 to 95.0 wt. %, more preferably 80.0 to 92.5 wt. % and in particular 82.5 to 90.0 wt. % of an aqueous carrier.

A preferred aqueous carrier includes exclusively water. Aqueous-alcoholic cosmetic carriers within the meaning of the present invention are to be understood as aqueous solutions that include 3 to 80.0 wt. % of a C₁-C₆ alcohol, in particular methanol, ethanol or propanol, isopropanol, butanol, isobutanol, tert.-butanol, n-pentanol, isopentanols, n-hexanol, isohexanols, glycol, glycerol, 1,2-pentanediol, 1,5-pentanediol, 1,2-hexanediol or 1,6-hexanediol and mixtures thereof. The agents according to the invention can additionally include other organic solvents, such as e.g. methoxybutanol, benzyl alcohol, ethyl diglycol or 1,2-propylene glycol. All water-soluble organic solvents are preferred here. More preferred, besides the compulsorily present water, are ethanol or propanol, isopropanol, butanol, isobutanol, tert.-butanol, n-pentanol, isopentanols, n-hexanol, isohexanols, 1,2-pentanediol, 1,5-pentanediol, 1,2-hexanediol or 1,6-hexanediol and mixtures thereof. Most preferred are those aqueous carriers which, in addition to water, include ethanol or propanol, isopropanol, butanol, isobutanol, tert.-butanol, n-pentanol, isopentanols, 1,2-pentanediol or 1,5-pentanediol and mixtures thereof. The best results are obtained if, in addition to the compulsory water, at least ethanol, propanol or isopropanol and mixtures thereof are used. Preferred aqueous carriers include at least 50 wt. % water.

Depending on the composition of the aqueous carrier, two embodiments of the present invention in particular can be implemented. The two embodiments are as follows:

### Embodiment 1

### Of the Invention

Water is used exclusively as the carrier in a quantity of at least 80.0 wt. %. Preferred agents according to the invention are characterized in that they include—based on their weight—80.0 to 98.0 wt. %, preferably 80.0 to 95.0 wt. %, more preferably 80.0 to 92.5 wt. % and in particular 82.5 to 90.0 wt. % water.

The inventors have found that the quantity of water of at least 80.0 wt. % must be precisely maintained since, with quantities below this, the composition no longer separates clearly and the formation of three separate phases, each of which is present as a separate, visible phase, no longer occurs.

In this embodiment, the three phases, viewed from bottom to top, are as follows: aqueous phase/silicone-containing phase/virgin oil phase.

### Embodiment 2

### Of the Present Invention

In this embodiment, an aqueous-alcoholic carrier is used. Aqueous-alcoholic cosmetic carriers within the meaning of the present invention are to be understood as aqueous solutions that include 3 to 80.0 wt. % of a C₁-C₆ alcohol, in particular methanol, ethanol or propanol, isopropanol, butanol, isobutanol, tert.-butanol, n-pentanol, isopentanols, n-hexanol, isohexanols, glycol, glycerol, 1,2-pentanediol, 1,5-pentanediol, 1,2-hexanediol or 1,6-hexanediol and mixtures thereof. The agents according to the invention can additionally include further organic solvents, such as e.g. methoxybutanol, benzyl alcohol, ethyl diglycol or 1,2-propylene glycol. All water-soluble organic solvents are preferred here. More preferred, besides the compulsorily present water, are ethanol or propanol, isopropanol, butanol, isobutanol, tert.-butanol, n-pentanol, isopentanols, n-hexanol, isohexanols, 1,2-pentanediol, 1,5-pentanediol, 1,2-hexanediol or 1,6-hexanediol and mixtures thereof. Most preferred are those aqueous carriers which, in addition to water, include ethanol or propanol, isopropanol, butanol, isobutanol, tert.-butanol, n-pentanol, isopentanols, 1,2-pentanediol or 1,5-pentanediol and mixtures thereof. The best results are obtained if in addition to the compulsory water, at least ethanol, propanol or isopropanol and mixtures thereof are used. More preferred aqueous carriers include at least 50 wt. % water.

In this embodiment, the three phases viewed from bottom to top are as follows: silicone-containing phase/virgin oil phase/aqueous-alcoholic phase.

The second compulsory constituent according to the invention is selected from the dimethicones and/or dimethiconols. The dimethicones according to the invention can be either linear or branched or cyclic, or cyclic and branched. Linear dimethicones can be represented by the following structural formula (Si1):

(SiR¹₃)—O—(SiR²₂—O—(SiR¹₃) (Si1)

Branched dimethicones can be represented by the structural formula (Si1.1):

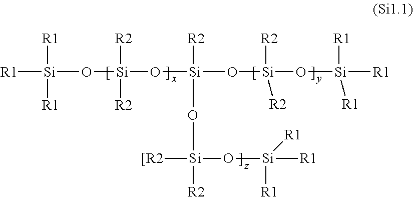

The residues R¹ and R² each independently of one another denote hydrogen, a methyl residue, a C2 to C30 linear, saturated or unsaturated hydrocarbon residue, a phenyl residue and/or an aryl residue. The numbers x, y and z are integers and each independently of one another run from 0 to 50,000. The molecular weights of the dimethicones are between 1000 D and 10,000,000 D. The viscosities are between 50 and 10,000,000 mm²/s measured at 25° C. using a glass capillary viscometer by the Dow Corning Corporate Test Method CTM 0004 of 20 Jul. 1970. Preferred viscosities are between 100 and 350 mm²/s. The product “Dow Corning 200 with a viscosity of 50 to 350 mm²/s” may be referred to here by way of example.

More preferred cosmetic or dermatological preparations according to the invention are characterized in that they include at least one silicone of the formula (Si1.2)

(CH₃)₃Si—[O—Si(CH₃)₂]ₓ—O—Si(CH₃)₃ (Si1.2),

in which x denotes a number from 0 to 100, preferably from 0 to 50, more preferably from 0 to 20 and in particular 0 to 10.

The dimethicones (Si1) are included in the compositions according to the invention in quantities of 0.01 to 10 wt. %, preferably 0.01 to 8 wt. %, more preferably 0.1 to 7.5 wt. % and in particular 0.1 to 5 wt. %, based on the total composition.

Finally, the silicone compounds are understood to include the dimethiconols (Si8). The dimethiconols according to the invention can be either linear or branched or cyclic, or cyclic and branched. Linear dimethiconols can be illustrated by the following structural formula (Si8-I):

(SiOHR¹₂)—O—(SiR²₂—O—)ₓ—(SiOHR¹₂) (Si8-I)

Branched dimethiconols can be illustrated by the structural formula (Si8-II):

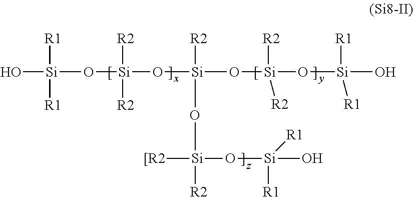

The residues R¹ and R² each independently of one another denote hydrogen, a methyl residue, a C2 to C30 linear, saturated or unsaturated hydrocarbon residue, a phenyl residue and/or an aryl residue. The numbers x, y and z are integers and each independently of one another run from 0 to 50,000. The molecular weights of the dimethiconols are between 1000 D and 10,000,000 D. The viscosities are between 100 and 350 mm²/s measured at 25° C. using a glass capillary viscometer by the Dow Corning Corporate Test Method CTM 0004 of 20 Jul. 1970.

The density of the dimethicones and/or dimethiconols according to the invention, measured at 25° C., is between 0.95 g/cm³ and 0.98 g/cm³, preferably between 0.955 g/cm³ and 0.975 g/cm³, more preferably between 0.96 g/cm³ and 0.975 g/cm³ and most preferably between 0.965 g/cm³ and 0.975 g/cm³. While it is true that cyclomethicones can also be used according to the invention, it is essential to ensure that their density lies within the range stated above. Often, the density of cyclomethicones specifically is less than 0.95 g/cm³ and therefore too low according to the invention.

If dimethicones and/or dimethiconols with a viscosity lower than 100 mm²/s are used, they mix too strongly with the oils. This results in insufficient phase separation from the phase that includes the oil.

If dimethicones and/or dimethiconols with a viscosity greater than 350 mm²/s are used, the three separate phases cannot be mixed by simply shaking the total composition by hand to form a no briefly homogeneous single-phase composition for application.

The agents according to the invention include at least 0.1 to 15 wt % of dimethicones and/or dimethiconols. More preferred agents according to the invention are characterized in that they include—based on their weight—0.5 to 10.0 wt. %, preferably 1.0 to 9 wt. %, more preferably 2.0 to 8.5 wt. %, still more preferably 2.5 to 8.0 wt. %, still more preferably 3.0 to 7.5 wt. % and in particular 3.5 to 7.5 wt. % dimethicones and/or dimethiconols.

The third compulsory component of the present invention is an oil.

These oils include within the meaning of the present invention e.g.:

Vegetable oils, such as acai oil, algae oil, amaranth seed oil, anise oil, annatto oil, apricot kernel oil, apple seed oil, argan oil, avellana oil, avocado oil, babacu oil, babassu oil, baobab oil, cottonseed oil, bitter almond oil, borage seed oil, broccoli seed oil, camelina oil, cashew oil, cupuacu oil, safflower oil, peanut oil, eucalyptus oil, fennel oil, fish oil, pomegranate seed oil, grapefruit seed oil, rosehip seed oil, hemp oil, hazelnut oil, raspberry seed oil, elderberry seed oil, honeydew melon oil, jatropha oil, blackcurrant seed oil, St. John's wort oil, jojoba oil, cocoa butter, cocoa oil, coffee oil, camellia oil, cherry kernel oil, coconut oil, pumpkin seed oil, salmon oil, flax oil, linseed oil, gold-of-pleasure oil, bay oil, macadamia nut oil, maize germ oil, almond oil, mango butter, passion fruit oil, manila oil, melon oil, poppy oil, evening primrose oil, mink oil, olive oil, olive seed oil, palm oil, palm kernel oil, papaya seed oil, patchouli oil, peach kernel oil, plum kernel oil, pecan nut oil, perilla oil, pistachio oil, cranberry seed oil, rambutan oil, rapeseed oil, rice germ oil, rice oil, castor oil, sacha inchi oil, sea buckthorn berry oil, sea buckthorn seed oil, black cumin oil, mustard oil, sesame oil, shea butter, soybean oil, sunflower oil, squalone oil, sweet almond oil, tea tree oil, thyme oil, grape seed oil, tung oil, ucuuba butter, vassoura oil, walnut oil, watermelon oil, wheat germ oil, cuckoo flower oil, wild rose oil, yak hair oil, ylang-ylang oil or civet oil. This list is to be understood as being by way of example. Any natural oil, in particular any oil that is known as a nutrient or from the naturopathy sector, can be a constituent of the composition according to the invention. Furthermore, a mixture of any of the above-mentioned oils can also be used according to the invention. Preferred oils according to the invention are selected from apricot kernel oil, apple seed oil, argan oil, babassu oil, cottonseed oil, bitter almond oil, cashew oil, cupuacu oil, safflower oil, peanut oil, pomegranate seed oil, grapefruit seed oil, rosehip seed oil, hemp oil, hazelnut oil, raspberry seed oil, elderberry seed oil, honeydew melon oil, blackcurrant seed oil, jojoba oil, cocoa butter, cocoa oil, coffee oil, camellia oil, cherry kernel oil, coconut oil, pumpkin seed oil, bay oil, macadamia nut oil, maize germ oil, almond oil, mango butter, passion fruit oil, marula oil, melon oil, poppy oil, evening primrose oil, olive oil, olive seed oil, papaya seed oil, patchouli oil, peach kernel oil, plum kernel oil, pecan nut oil, perilla oil, pistachio oil, cranberry seed oil, rice germ oil, rice oil, castor oil, sacha inchi oil, sea buckthorn berry oil, sea buckthorn seed oil, black cumin oil, mustard oil, sesame oil, shea butter, soybean oil, sunflower oil, squalone oil, sweet almond oil, tea tree oil, grape seed oil, ucuuba butter, walnut oil, watermelon oil, wheat germ oil, cuckoo flower oil, wild rose oil and the liquid fractions of coconut oil. More preferred oils are selected from apricot kernel oil, argan oil, babassu oil, cottonseed oil, bitter almond oil, cashew oil, pomegranate seed oil, grapefruit seed oil, rosehip seed oil, hazelnut oil, raspberry seed oil, elderberry seed oil, blackcurrant seed oil, jojoba oil, cherry kernel oil, coconut oil, bay oil, macadamia nut oil, almond oil, mango butter, passion fruit oil, marula oil, evening primrose oil, olive oil, olive seed oil, patchouli oil, peach kernel oil, plum kernel oil, pecan nut oil, pistachio oil, sacha inchi oil, sea buckthorn berry oil, sea buckthorn seed oil, sesame oil, shea butter, sweet almond oil, tea tree oil, grape seed oil, walnut oil, wild rose oil and the liquid fractions of coconut oil and mixtures thereof.

However, other triglyceride oils, such as the liquid portions of beef tallow and synthetic triglyceride oils, are also suitable.

Likewise, liquid paraffin oils, isoparaffin oils and synthetic hydrocarbons as well as di-n-alkyl ethers with a total of between 12 and 36 C atoms, in particular 12 to 24 C atoms, such as e.g. di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether as well as di-tert-butyl ether, diisopentyl ether, di-3-ethyldecyl ether, tert.-butyl-n-octyl ether, isopentyl-n-octyl ether and 2-methylpentyl-n-octyl ether as well as the compounds 1,3-di-(2-ethylhexyl) cyclohexane (Cetiol® S) and di-n-octyl ether (Cetiol® OE), which are available as commercial products, can be suitable.

Finally, ester oils are also suitable oils according to the invention. Ester oils are to be understood as the esters of C6-C30 fatty acids with C2-C30 fatty alcohols. Preferred are the monoesters of fatty acids with alcohols having 2 to 24 C atoms. Examples of fatty acid portions employed in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidic acid, gadoleic acid, behenic acid and erucic acid as well as technical mixtures thereof. Examples of the fatty alcohol portions in the ester oils are isopropyl alcohol, caproyl alcohol, capryl alcohol, 2-ethylhexyl alcohol, capric alcohol, lauryl alcohol, isotridecyl alcohol, myristyl alcohol, cetyl alcohol, palmitoleyl alcohol, stearyl alcohol, isostearyl alcohol, oleyl alcohol, elaidyl alcohol, petroselinyl alcohol, linoleyl alcohol, linolenyl alcohol, elaeostearyl alcohol, arachidyl alcohol, gadoleyl alcohol, behenyl alcohol, erucyl alcohol and brassidyl alcohol as well as technical mixtures thereof. More preferred according to the invention are isopropyl myristate (Rilanit® IPM), isononanoic acid C16-18 alkyl esters (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V).

Another group of suitable oils according to the invention are dicarboxylic acid esters such as di-n-butyl adipate, di(2-ethylhexyl) adipate, di(2-ethylhexyl) succinate and diisotridecyl acetate as well as diol esters such as ethylene glycol dioleate, ethylene glycol diisotridecanoate, propylene glycol di(2-ethylhexanoate), propylene glycol diisostearate, propylene glycol dipelargonate, butanediol diisostearate, neopentyl glycol dicaprylate.

Finally, symmetrical, asymmetrical or cyclic esters of carbonic acid with fatty alcohols, e.g. glycerol carbonate or dicaprylyl carbonate (Cetiol® CC), are also suitable according to the invention.

The last group of oils that are suitable according to the invention are mono-, di- and trifatty acid esters of saturated and/or unsaturated, linear and/or branched fatty acids with glycerol, such as e.g. Monomuls® 90-018, Monomuls® 90-L12 or Cutina® MD.

The oils are preferably selected from the vegetable oils and the ester oils and mixtures thereof.

One criterion for the selection of an oil according to the invention is the density of the oil at 25° C. The density of the oil should be 0.75 to 0.95 g/cm³. The density of the oil is preferably 0.8 to 0.94 g/cm³ and more preferably 0.85 to 0.93 g/cm³.

The quantity employed is 0.1-15 wt. %, based on the total agent, preferably 0.1-10 wt. % and more preferably 0.1-15 wt. %, based on the total agent.

The agents according to the invention include at least 0.1 to 15 wt. % of the oils described above. More preferred agents according to the invention are characterized in that they include—based on their weight—0.5 to 10.0 wt. %, preferably 1.0 to 9 wt. %, more preferably 2.0 to 8.5 wt. %, still more preferably 2.5 to 8.0 wt. %, still more preferably 3.0 to 7.5 wt. % and in particular 3.5 to 7.5 wt. % oils.

The ingredients b) and c) of the present invention are used in a ratio of 5:1 to 1:5, preferably 3:1 to 1:3, more preferably in a ratio of 2:1 to 1:2 and most preferably in a ratio of 1:1.

Even though the three phases of the present invention should be present separately, with visible separation, they must nevertheless be capable of being converted to a briefly homogeneous composition immediately before application by simply shaking the composition in the selected outer package, preferably a transparent container.

Products of this type possess high optical differentiation and good consumer acceptance. The consumer shakes the product before application and applies the briefly stable emulsion onto the hair, while the rest of the emulsion separates again in the product package. The briefly stable emulsion here should be stable for a period of a few seconds up to a maximum of 10 minutes, preferably 30 sec to 5 minutes. After application, the three-phase nature should be re-established within a few minutes, so that the consumer realizes that, if necessary, he must homogenize the product again by shaking before further application. If the originally optically three-phase composition is left to stand again in its ideally transparent container at room temperature (23 to 28° C.), the three optically separate and clearly distinguishable phases form again after a standing time of a few minutes and at most 48 hours, preferably after 1 hour to 24 hours, more preferably after 1 hour to 12 hours.

It can be preferred according to the invention if each of the three phases is colored with a color of its own. It is, of course, also included that only the middle phase is colored while the other two phases remain clear and transparent. However, it is also possible for the top and bottom phases to be colored while the middle phase remains clear and transparent. Finally, it is also to be understood as coloring if at least one phase includes an opacifying agent or a pearl luster pigment. These ingredients are very well known to the person skilled in the art. Finally, at least one of the three phases can also be an emulsion in itself.

Further ingredients are therefore also possible in the composition according to the invention.

The agents according to the invention furthermore contain, if desired, at least one silicone-based water-in-oil emulsifier from the group of the C8-C30 alkyl PEG/PPG dimethicones, the former INCI name of which was dimethicone copolyol, with the current INCI names C8-C30 PEG-x dimethicone (with x=2-20, preferably 3-17, more preferably 7-12), PEG/PPG a/b dimethicone (wherein a and b are independent of one another and a denotes numbers from 2-30, preferably 3-30 and more preferably 5-20, in particular 7-18, and b denotes numbers from 0-30, preferably 0 to 20 and more preferably from 0 to 15 and in particular from 0 to 12). Within the group of the C8-C30 alkyl PEG/PPG dimethicones, those which carry an alkyl group of C8 to C22, more preferably of C12 to C22 and in particular of C12 to C18 are preferred. The most preferred C8-C30 alkyl PEG/PPG dimethicones are lauryl, myristyl, cetyl and stearyl PEG/PPG dimethicones. These most preferred dimethicones include as PEG/PPG a/b dimethicones for a and b each independently of one another with numbers for a from 2-30, preferably 3-30 and more preferably 5-20, in particular 7-18, and b for numbers from 0-30, preferably 0 to 20 and more preferably from 0 to 15 and in particular from 0 to 12. Most preferred are e.g. the commercial products Abil® EM-90, Microcare Silicone E 1016 (Thor), Dow Corning® Q2-5200 or mixtures thereof.

Preferred agents according to the invention include silicone-based water-in-oil emulsifiers from the group of the C8-C30 alkyl PEG/PPG dimethicones preferably within relatively narrow quantitative ranges. In this case, agents according to the invention are preferred which include—based on their weight—0.15 to 1.25 wt. %, preferably 0.2 to 1.0 wt. %, more preferably 0.25 to 0.75 wt. % and in particular 0.3 to 0.5 wt. % of at least one silicone-based water-in-oil emulsifier from the group of the C8-C30 alkyl PEG/PPG dimethicones.

Cyclic siloxanes, the cyclic dimethicones designated as cyclomethicones according to INCI, can be employed according to the invention. In this case, cosmetic or dermatological preparations according to the invention are preferred which include at least one silicone of the formula (Si-4)

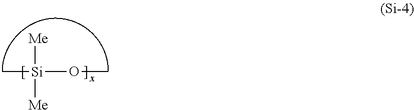

in which x denotes a number from 3 to 200, preferably from 3 to 10, more preferably from 3 to 7 and in particular 3, 4, 5 or 6. Most preferred, with x=5, is cyclopentasiloxane (CAS 2,4,6,8,10-pentamethylcyclopentasiloxane).

The agents according to the invention include at least 0.1 to 10 wt. % of the cyclic siloxanes. More preferred agents according to the invention are characterized in that they include—based on their weight—0.5 to 9.5 wt. %, preferably 1.0 to 9 wt. %, more preferably 2.0 to 8.5 wt. %, still more preferably 2.5 to 8.0 wt. %, still more preferably 3.0 to 7.5 wt. % and in particular 3.5 to 7.5 wt. % cyclic siloxanes.

In addition to the silicone-based water-in-oil emulsifier from the group of the C8-C30 alkyl PEG/PPG dimethicones, the agents according to the invention include, if desired, 0.01 to 5 wt. % of at least one silicone-free emulsifier.

Preferred oil-in-water emulsifiers according to the invention have an HLB value of at least 8, wherein the total oil-in-water emulsifier system preferably has a weight average (weight averaged) HLB value in the range of 11-17, preferably 13.5 to 15.5. These are emulsifiers that are generally known to the person skilled in the art, as listed e.g. in Kirk-Othmer, “Encyclopedia of Chemical Technology”, 3^{rd} edition, 1979, volume 8, pages 913-916. For ethoxylated products, the HLB value is calculated by the formula HLB=(100−L):5, wherein L is the proportion by weight of lipophilic groups, i.e. fatty alkyl or fatty acyl groups, in the ethylene oxide adducts, expressed as a percentage by weight.

When selecting suitable silicone-free oil-in-water emulsifiers according to the invention, preferably non-ionic oil-in-water emulsifiers, it is more preferred to employ a mixture of oil-in-water emulsifiers, preferably non-ionic oil-in-water emulsifiers, in order to be able to adjust the stability of the compositions according to the invention to the optimum level. The individual emulsifier components provide a proportion of the total HLB value or average HLB value of the oil-in-water emulsifier mixture in accordance with their proportion by weight of the total weight of the oil-in-water emulsifiers. Preferably according to the invention, the weight average HLB value of the oil-in-water emulsifier system is 11-17, preferably 12-15 and more preferably 13.5 to 15.5. To achieve these HLB values, preferably oil-in-water emulsifiers from the HLB value ranges of 10-14, 14-16 and optionally 15-17 can be combined with one another. Of course, the oil-in-water emulsifier mixtures (or oil-in-water emulsifier systems) can also include emulsifiers, preferably non-ionic emulsifiers, with HLB values in the range of >7-10 and 17-20; emulsifier mixtures of this type can likewise be preferred according to the invention. However, in another preferred embodiment the compositions according to the invention can also include only a single oil-in-water emulsifier with an HLB value in the range of 11-17, preferably 12-15 and more preferably 13-14.

Preferred agents according to the invention are characterized in that the silicone-free oil-in-water emulsifiers are selected from ethoxylated C₈-C₂₄ alkanols with on average 8-100 moles ethylene oxide per mole, ethoxylated C₈-C₂₄ carboxylic acids with on average 8-100 moles ethylene oxide per mole, glycerol mono- and/or diesters of linear saturated and unsaturated C₁₂-C₃₀ carboxylic acids, ethoxylated with on average 20-100 moles ethylene oxide per mole, which can be hydroxylated, in particular those of myristic acid, palmitic acid, stearic acid, 12-hydroxystearic acid or of mixtures of these fatty acids, sorbitan monoesters of linear saturated and unsaturated C12-C30 carboxylic acids ethoxylated with on average 20-100 moles ethylene oxide per mole, which can be hydroxylated, in particular those of myristic acid, palmitic acid, stearic acid, 12-hydroxystearic acid or of mixtures of these fatty acids, silicone copolyols with ethylene oxide units or with ethylene oxide and propylene oxide units, alkyl mono- and oligoglycosides with 8 to 22 carbon atoms in the alkyl residue and ethoxylated analogs thereof, ethoxylated sterols, partial esters of polyglycerols with n=2 to 10 glycerol units and esterified with 1 to 4 saturated or unsaturated, linear or branched, optionally hydroxylated C₈-C₃₀ fatty acid residues, provided that they have an HLB value of more than 7, as well as mixtures of the aforementioned substances.

The ethoxylated C₈-C₂₄ alkanols have the formula R¹O(CH₂CH₂O)ₙH, wherein R¹ denotes a linear or branched alkyl and/or alkenyl residue with 8-24 carbon atoms and n denotes the average number of ethylene oxide units per molecule, for numbers from 8-100, preferably 8-30 moles ethylene oxide to 1 mole capryl alcohol, 2-ethylhexyl alcohol, capric alcohol, lauryl alcohol, isotridecyl alcohol, tridecyl alcohol, myristyl alcohol, cetyl alcohol, palmitoleyl alcohol, stearyl alcohol, isostearyl alcohol, oleyl alcohol, elaidyl alcohol, petroselinyl alcohol, arachidyl alcohol, gadoleyl alcohol, behenyl alcohol, erucyl alcohol and brassidyl alcohol as well as technical mixtures thereof. Adducts of 8-100 moles ethylene oxide to technical fatty alcohols with 12-18 carbon atoms, such as e.g. coconut, palm, palm kernel or tallow fatty alcohol, are also suitable.

The ethoxylated C₈-C₂₄ carboxylic acids have the formula R¹O(CH₂CH₂O)ₙH, wherein R¹O denotes a linear or branched saturated or unsaturated acyl residue with 8-24 carbon atoms and n denotes the average number of ethylene oxide units per molecule, for numbers from 8-100, preferably 10-30 moles ethylene oxide to 1 mole caprylic acid, 2-ethylhexanoic acid, capric acid, lauric acid, isotridecanoic acid, myristic acid, cetylic acid, palmitoleic acid, stearic acid, isostearic acid, oleic acid, elaidic acid, petroselic acid, arachidic acid, gadoleic acid, behenic acid, erucic acid and brassidic acid as well as technical mixtures thereof. Adducts of 10-100 moles ethylene oxide to technical fatty acids with 12-18 carbon atoms, such as coconut, palm, palm kernel or tallow fatty acid, are also suitable. More preferred are PEG-50 monostearate, PEG-100 monostearate, PEG-50 monooleate, PEG-100 monooleate, PEG-50 monolaurate and PEG-100 monolaurate.

More preferably, the C₁₂-C₁₈ alkanols or the C₁₂-C₁₈ carboxylic acids with in each case 8-30 units ethylene oxide per molecule as well as mixtures of these substances, in particular laureth-8, laureth-10, laureth-12, laureth-20, trideceth-8, trideceth-9, trideceth-10, trideceth-12, trideceth-20, ceteth-10, ceteth-12, ceteth-20, ceteth-30, steareth-10, steareth-12, steareth-20, steareth-30, ceteareth-10, ceteareth-12, ceteareth-20, ceteareth-30, laureth-12 and beheneth-20, are employed.

Preferred glycerol mono- and/or diesters of linear saturated and unsaturated C₁₂-C₃₀ carboxylic acids ethoxylated with on average 20-100 moles ethylene oxide per mole, which can be hydroxylated, are selected from PEG-20 hydrogenated castor oil, PEG-40 hydrogenated castor oil and PEG-60 hydrogenated castor oil.

Preferred sorbitan monoesters of linear saturated and unsaturated C₁₂-C₃₀ carboxylic acids ethoxylated with on average 20-100 moles ethylene oxide per mole, which can be hydroxylated, are selected from polysorbate-20, polysorbate-40, polysorbate-60 and polysorbate-80.

Furthermore, C₈-C₂₂ alkyl mono- and oligoglycosides are preferably employed. C₈-C₂₂ alkyl mono- and oligoglycosides represent known, commercially available surfactants and emulsifiers. They are produced in particular by reacting glucose or oligosaccharides with primary alcohols having 8-22 carbon atoms. With regard to the glycoside residue, both monoglycosides in which a cyclic sugar residue is bound glycosidically to the fatty alcohol and oligomeric glycosides with a degree of oligomerization of up to about 8, preferably 1-2, are suitable. The degree of oligomerization here is a statistical average, which is based on a conventional homolog distribution for these technical products. Products that are available with the trade mark Plantacare® include a glucosidically bound C₈-C₁₆ alkyl group on an oligoglucoside residue, the average degree of oligomerization of which is 1-2, in particular 1.2-1.4. More preferred C₈-C₂₂ alkyl mono- and oligoglycosides are selected from octyl glucoside, decyl glucoside, lauryl glucoside, palmityl glucoside, isostearyl glucoside, stearyl glucoside, arachidyl glucoside and behenyl glucoside as well as mixtures thereof. The acyl glucamides that are derived from glucamine are also suitable as non-ionic oil-in-water emulsifiers.

Ethoxylated sterols, in particular ethoxylated soy sterols, also represent suitable oil-in-water emulsifiers according to the invention. The degree of ethoxylation must be greater than 5, preferably at least 10, in order to have an HLB value greater than 7. Suitable commercial products are e.g. PEG-10 Soy Sterol, PEG-16 Soy Sterol and PEG-25 Soy Sterol.

Furthermore, partial esters of polyglycerols with 2 to 10 glycerol units and esterified with 1 to 4 saturated or unsaturated, linear or branched, optionally hydroxylated C8-C30 fatty acid residues are preferably employed, provided that they have an HLB value of more than 7. More preferred are diglycerol monocaprylate, diglycerol monocaprate, diglycerol monolaurate, triglycerol monocaprylate, triglycerol monocaprate, triglycerol monolaurate, tetraglycerol monocaprylate, tetraglycerol monocaprate, tetraglycerol monolaurate, pentaglycerol monocaprylate, pentaglycerol monocaprate, pentaglycerol monolaurate, hexaglycerol monocaprylate, hexaglycerol monocaprate, hexaglycerol monolaurate, hexaglycerol monomyristate, hexaglycerol monostearate, decaglycerol monocaprylate, decaglycerol monocaprate, decaglycerol monolaurate, decaglycerol monomyristate, decaglycerol monoisostearate, decaglycerol monostearate, decaglycerol monooleate, decaglycerol monohydroxystearate, decaglycerol dicaprylate, decaglycerol dicaprate, decaglycerol dilaurate, decaglycerol dimyristate, decaglycerol diisostearate, decaglycerol distearate, decaglycerol dioleate, decaglycerol dihydroxystearate, decaglycerol tricaprylate, decaglycerol tricaprate, decaglycerol trilaurate, decaglycerol trimyristate, decaglycerol triisostearate, decaglycerol tristearate, decaglycerol trioleate and decaglycerol trihydroxystearate.

Of course, instead of or in addition to non-ionic emulsifiers, ionic silicone-free emulsifiers can also be employed. In particular, cationic emulsifiers are preferred here, see below.

Regardless of the nature of the silicone-free emulsifier, agents according to the invention are preferred which include the silicone-free emulsifiers within relatively narrow quantitative ranges. These preferred agents are characterized in that they include—based on their weight—0.02 to 4.5 wt. %, preferably 0.03 to 4.0 wt. %, more preferably 0.04 to 3.5 wt. %, still more preferably 0.05 to 3.0 wt. % and in particular 0.1 to 1.0 wt. % of at least one silicone-free water-in-oil emulsifier.

As already mentioned, cationic compounds in particular are preferred as silicone-free emulsifiers. In this case, agents according to the invention are preferred which include exclusively cationic compounds as silicone-free emulsifiers.

By employing quaternary surface-active compounds with an antimicrobial action, the agent can be provided with an antimicrobial action or, if applicable, its existing antimicrobial action based on other ingredients can be enhanced. Suitable QACs for this purpose are e.g. benzalkonium chloride (N-alkyl-N,N-dimethylbenzylammonium chloride, CAS no. 8001-54-5), Benzalkon B (m,p-dichlorobenzyldimethyl-C₁₂-alkylammonium chloride, CAS no. 58390-78-6), benzoxonium chloride (benzyldodecyl-bis(2-hydroxyethyl)ammonium chloride), cetrimonium bromide (N-hexadecyl-N,N-trimethylammonium bromide, CAS no. 57-09-0), benzethonium chloride (N,N-dimethyl-N-[2-[2-[p-(1,1,3,3-tetramethylbutyl)phenoxy]ethoxy]ethyl]benzylammonium chloride, CAS no. 121-54-0), dialkyldimethylammonium chlorides, such as di-n-decyldimethylammonium chloride (CAS no. 7173-51-5-5), didecyldimethylammonium bromide (CAS no. 2390-68-3), dioctyldimethylammonium chloride, 1-cetylpyridinium chloride (CAS no. 123-03-5) and thiazoline iodide (CAS no. 15764-48-1) and mixtures thereof. Preferred. QACs are the benzalkonium chlorides with C₈-C₁₈ alkyl residues, in particular C12-C14 alkylbenzyldimethylammonium chloride. A more preferred QAC is coco pentaethoxymethylammonium methosulfate (INCI PEG-5 Cocomonium methosulfate; Rewoquat® CPEM).

Furthermore, for preference, at least one quaternary imidazoline compound, i.e. a compound having a positively charged imidazoline ring, is included as cationic surfactant. Formula I illustrated below shows the structure of these compounds.

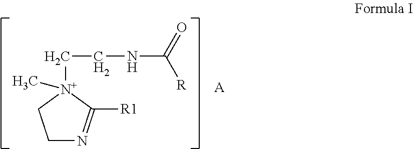

The residues R and R1 each independently of one another denote a saturated or unsaturated, linear or branched hydrocarbon residue with a chain length of 8 to 30 carbon atoms. The preferred compounds of formula I include the same hydrocarbon residue for each of R and R1. The chain length of the residues R and R1 is preferably 12 carbon atoms. More preferred are compounds with a chain length of at least 16 carbon atoms and particularly preferably with at least 20 carbon atoms. A most preferred compound of formula I has a chain length of 21 carbon atoms. A product with this chain length is known e.g. with the name quaternium-91 or the trade names Crodazosoft® DBQ, which also includes cetrimonium methosulfate and cetearyl alcohol besides quarternium-91, and Crodazosoft® SCQ, which also includes PPG-3 benzyl ether myristate besides quaternium-91. Examples that are particularly in accordance with the invention are available e.g. with the INCI names quaternium-27, quaternium-72, quaternium-83 and quaternium-91. The commercial products Crodazosoft® DBQ and Crodazosoft® SCQ, or quaternium-91, are most preferably used.

The imidazolines of formula I are included in the compositions according to the invention in quantities of 0.01 to 20 wt. %, preferably in quantities of 0.05 to 10 wt. % and particularly preferably in quantities of 0.1 to 7.5 wt. %. The best results of all are obtained here with quantities of 0.1 to 5 wt. %, based in each case on the total composition of the respective agent.

Furthermore, the following cationic surfactants according to the formula (Tkat-2) can also be used.

RCO—X—N⁺R¹R²R³A⁻ (Tkat-2)

R here denotes a substituted or unsubstituted, branched or straight-chained alkyl or alkenyl residue with 11 to 35 carbon atoms in the chain,

X denotes —O— or —NR⁵,
R¹ denotes an alkylene group with 2 to 6 C atoms, which can be unsubstituted or substituted, wherein in the case of a substitution, substitution with an —OH or —NH group is preferred,
R², R³ each independently of one another denote an alkyl or hydroxyalkyl group with 1 to 6 C atoms in the chain, wherein the chain can be linear or branched,
R5 denotes hydrogen or a C1 to C6 straight-chained or branched, alkyl or alkenyl residue, which can also be substituted by a hydroxy group.

Within this class of structures, the compounds of one of the following structures are preferably used:

CH₃(CH₂)₂₀CONH(CH₂)₃—N⁺(CH₃)₂—CH₂CH₃A (Tkat-3)

CH₃(CH₂)₂₀CONH(CH₂)₃—N⁺(CH₃)₂—CH₂(CHOH)CH₂OH A (Tkat-4)

CH₃(CH₂)₂₀COOCH₂CHOHCH₂—N⁺(CH₃)₃A (Tkat-5)

CH₃(CH₂)₂₀CONH(CH₂)₃—N⁺(CH₃)₂—CH₂CH₂OH A (Tkat-6)

Examples of commercial products of this type are Schercoquat BAS, Lexquat AMG-BEO, Akypoquat 131 or Incroquat Behenyl HE.

Furthermore, esterquats according to the formula (Tkat1-2) can be used.

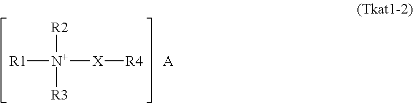

The residues R1, R2 and R3 are each independent of one another here and can be the same or different. The residues R1, R2 and R3 signify:
a branched or unbranched alkyl residue with 1 to 4 carbon atoms, which can include at least one hydroxyl group, or
a saturated or unsaturated, branched or unbranched or a cyclic saturated or unsaturated alkyl residue with 6 to 30 carbon atoms, which can include at least one hydroxyl group, or
an aryl or alkaryl residue, e.g. phenyl or benzyl,
the residue (—X—R4), with the proviso that no more than 2 of the residues R1, R2 or R3 can denote this residue.

The residue —(X—R4) is included at least 1 to 3 times.

X here denotes:
1) —(CH₂)n- with n=1 to 20, preferably n=1 to 10 and more preferably n=1-5, or
2) —(CH₂—CHR5-O)n- with n=1 to 200, preferably 1 to 100, more preferably 1 to 50, and more preferably 1 to 20 with R5 in the meaning of hydrogen, methyl or ethyl,
   and R4 denotes:
1) R6-O—CO—, where R6 is a saturated or unsaturated, branched or unbranched or a cyclic saturated or unsaturated alkyl residue with 6 to 30 carbon atoms, which can include at least one hydroxy group and which can optionally furthermore be ethoxylated with 1 to 100 ethylene oxide units and/or 1 to 100 propylene oxide units, or
2) R7-CO—, where R7 is a saturated or unsaturated, branched or unbranched or a cyclic saturated or unsaturated alkyl residue with 6 to 30 carbon atoms, which can include at least one hydroxy group and which can optionally furthermore be ethoxylated with 1 to 100 ethylene oxide units and/or 1 to 100 propylene oxide units,
   and A denotes a physiologically acceptable organic or inorganic anion.

Products of this type are marketed e.g. with the trademarks Rewoquat®, Stepantex®, Dehyquart® and Armocare®. The products Armocare®VGH-70, an N,N-bis(2-palmitoyloxyethyDdimethylammonium chloride, and Dehyquart® F-75, Dehyquart® C-4046, Dehyquart® L80, Dehyquart® F-30, Dehyquart® AU-35, Rewoquat® WE18, Rewoquat® WE38 DPG and Stepantex® VS 90 are examples of these esterquats.

Other more preferred compounds of the formula (Tkat1-2) according to the invention are included in the formula (Tkat1-2.1), the cationic betaine esters.

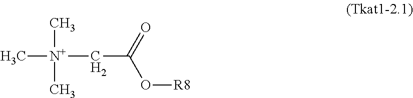

R8 corresponds in its meaning to R7.

As a further ingredient, monoalkyltrimethylammonium salts with a chain length of the alkyl residue of 16 to 24 carbon atoms can be included.

These compounds have the structure illustrated in the formula (Tkat1-1),

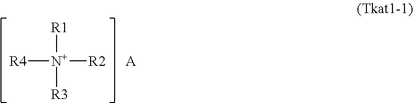

wherein R1, R2 and R3 each denote a methyl group and R4 denotes a saturated, branched or unbranched alkyl residue with a chain length of 16 to 24 carbon atoms. Examples of compounds of the formula (Tkat1-1) are cetyltrimethylammonium chloride, cetyltrimethylammonium bromide, cetyltrimethylammonium methosulfate, stearyltrimethylammonium chloride, behenyltrimethylammonium chloride, behenyltrimethylammonium bromide and behenyltrimethylammonium methosulfate.

In a more preferred embodiment of the invention, the agents according to the invention furthermore include at least one amine and/or cationized amine, in particular an amidoamine and/or a cationized amidoamine with the following structural formulae:

[R¹—NH—(CH₂)ₙ—NR²R³]A (Tkat7) and/or

[R¹—NH—(CH₂)ₙ—NR²R³R⁴]A (Tkat8)

where R1 signifies an acyl or alkyl residue with 6 to 30 C atoms, which can be branched or unbranched, saturated or unsaturated, and wherein the acyl residue and/or the alkyl residue can include at least one OH group, and
R2, R3 and R4 each independently of one another signify hydrogen or an alkyl residue with 1 to 4 C atoms, which can be the same or different, saturated or unsaturated, and
A signifies an anion and
n signifies an integer between 1 and 10.

Preferred is a composition in which the amine and/or the quaternized amine according to the general formulae (Tkat7) and/or (Tkat8) is an amidoamine and/or a quaternized amidoamine, where R1 signifies a branched or unbranched, saturated or unsaturated acyl residue with 6 to 30 C atoms, which can include at least one OH group. Preferred here is a fatty acid residue from oils and waxes, in particular from natural oils and waxes. As examples of these, lanolin, beeswax or candellila wax are suitable.

Preferred are also those amidoamines and/or quaternized amidoamines in which R2, R3 and/or R4 in the formulae (Tkat7) and/or (Tkat8) signify a residue according to the general formula CH₂CH₂OR5, where R5 can have the meaning of alkyl residues with 1 to 4 carbon atoms, hydroxyethyl or hydrogen. The preferred value of n in the general formulae (Tkat7) and/or (Tkat8) is an integer between 2 and 5.

The alkyl residue with 1 to 4 carbon atoms in R2, R3 and R4 in the general formula (Tkat7) and/or (Tkat8) can include at least one hydroxyl group.

The alkylamidoamines can either be present as such or can be converted to a quaternary compound in the composition by protonation in an appropriately acidic solution. Preferred according to the invention are the cationic alkylamidoamines.

Suitable amidoamines to be used according to the invention, which may optionally be quaternized, are for example the amidoamines: Witcamine 100 (Witco, INCI name: cocamidopropyl dimethylamine), Incromine BB (Croda, INCI name: behenamidopropyl dimethylamine), Mackine 401 (McIntyre, INCI name: isostearylamidopropyl dimethylamine) and other Mackine grades, Adogen S18V (Witco, INCI name: stearylamidopropyl dimethylamine), and as permanently cationic aminoamines: Rewoquat RTM 50 (Witco Surfactants GmbH, INCI name: ricinoleamidopropyltrimonium methosulfate), Empigen CSC (Albright & Wilson, INCI name: cocamidopropyltrimonium chloride), Swanol Lanoquat DES-50 (Nikko, INCI name: quaternium-33), Rewoquat UTM 50 (Witco Surfactants GmbH, undecyleneamidopropyltrimonium methosulfate).

The anion A according to all of the structural formulae listed above of all of the cationic compounds listed above is selected from the physiologically acceptable anions. The halide ions, fluoride, chloride, bromide, sulfate of the general formula RSO₃⁻, where R has the meaning of saturated or unsaturated alkyl residues with 1 to 4 carbon atoms, or anionic residues of organic acids such as maleate, fumarate, oxalate, tartrate, citrate, lactate or acetate, may be mentioned as examples of these.

The above-mentioned cationic surfactants can be used individually or in any combinations with one another, with quantities of 0.01 to 20 wt. %, preferably in quantities of 0.01 to 10 wt. % and particularly preferably in quantities of 0.1 to 7.5 wt. % being included. The best results of all are obtained here with quantities of 0.1 to 5 wt. %, based in each case on the total composition of the respective agent.

Particularly preferred agents according to the invention are characterized in that they include as silicone-free emulsifiers at least one cationic surfactant, preferably at least one imidazolinium salt or at least one C₈₋₂₄ alkyltrimethylammonium salt, which more preferably include C₁₀₋₂₀ alkyltrimethylammonium salt and still more preferably C₁₂₋₁₈ alkyltrimethylammonium salts and in particular cetyltrimethylammonium chloride, or a mixture of at least one imidazolinium salt and a C₈₋₂₄ alkyl-trimethylammonium salt.

Furthermore, cationized protein hydrolyzates should be included as a more preferred further component in addition to the compulsory ingredients according to claim 1, wherein the basic protein hydrolyzate can originate from an animal, e.g. from collagen, milk or keratin, from a plant, e.g. from wheat, maize, rice, potatoes, soy, moringa or almonds, from marine life forms, e.g. from fish collagen or algae, or protein hydrolyzates obtained by biotechnology. Preferred are those cationic protein hydrolyzates of which the basic protein portion has a molecular weight of 100 to 25000 daltons, preferably 250 to 5000 daltons, most preferably 250 to 1000 daltons. Furthermore, cationic protein hydrolyzates are understood to include quaternized amino acids and mixtures thereof. The quaternization of the protein hydrolyzates or amino acids is often carried out using quaternary ammonium salts, such as e.g. N,N-dimethyl-N-(n-alkyl)-N-(2-hydroxy-3-chloro-n-propyl)ammonium halides. As typical examples of the cationic protein hydrolyzates and derivatives according to the invention, the products that are mentioned with the INCI names in the “International Cosmetic Ingredient Dictionary and Handbook”, (seventh edition 1997, The Cosmetic, Toiletry and Fragrance Association, 1101 17^{th} Street, N.W., Suite 300, Washington, D.C. 20036-4702) and that are commercially available may be mentioned: cocodimonium hydroxypropyl hydrolyzed collagen, cocodimonium hydroxypropyl hydrolyzed casein, cocodimonium hydroxypropyl hydrolyzed collagen, cocodimonium hydroxypropyl hydrolyzed hair keratin, cocodimonium hydroxypropyl hydrolyzed keratin, cocodimonium hydroxypropyl hydrolyzed rice protein, cocodimonium hydroxypropyl hydrolyzed soy protein, cocodimonium hydroxypropyl hydrolyzed wheat protein, hydroxypropyl arginine lauryl/myristyl ether HCl, hydroxypropyltrimonium gelatin, hydroxypropyltrimonium hydrolyzed casein, hydroxypropyltrimonium hydrolyzed collagen, hydroxypropyltrimonium hydrolyzed conchiolin protein, hydroxypropyltrimonium hydrolyzed keratin, hydroxypropyltrimonium hydrolyzed rice bran protein, hydroxypropyltrimonium hydrolyzed soy protein, hydroxypropyl hydrolyzed vegetable protein, hydroxypropyltrimonium hydrolyzed wheat protein, hydroxypropyltrimonium hydrolyzed wheat protein/siloxysilicate, laurdimonium hydroxypropyl hydrolyzed soy protein, laurdimonium hydroxypropyl hydrolyzed wheat protein, laurdimonium hydroxypropyl hydrolyzed wheat protein/siloxysilicate, lauryldimonium hydroxypropyl hydrolyzed casein, lauryldimonium hydroxypropyl hydrolyzed collagen, lauryldimonium hydroxypropyl hydrolyzed keratin, lauryldimonium hydroxypropyl hydrolyzed soy protein, steardimonium hydroxypropyl hydrolyzed casein, steardimonium hydroxypropyl hydrolyzed collagen, steardimonium hydroxypropyl hydrolyzed keratin, steardimonium hydroxypropyl hydrolyzed rice protein, steardimonium hydroxypropyl hydrolyzed soy protein, steardimonium hydroxypropyl hydrolyzed vegetable protein, steardimonium hydroxypropyl hydrolyzed wheat protein, steartrimonium hydroxyethyl hydrolyzed collagen, quaternium-76 hydrolyzed collagen, quaternium-79 hydrolyzed collagen, quaternium-79 hydrolyzed keratin, quaternium-79 hydrolyzed milk protein, quaternium-79 hydrolyzed soy protein, quaternium-79 hydrolyzed wheat protein.

Particularly preferred are the plant-based cationic protein hydrolyzates and derivatives and most preferred are those based on wheat, rice, maize, soy, almond or moringa. Cationic protein hydrolyzates based on wheat are most preferably used. The commercial products Gluadin® WQ, Gluadin® WQT and the products from the Hydrotriticum® series from Croda are examples of these most preferred cationic protein hydrolyzates.

The cationic protein hydrolyzates are included in the compositions according to the invention preferably in quantities of 0.1 to 5.0 wt. %, based on the total agent. Quantities of 0.1 to 3 wt. % are more preferred.

The care effects of the agents according to the invention can be further enhanced by selecting a suitable pH value for the agents according to the invention. In particular, acidic agents according to the invention display extraordinarily good care properties without weighing down the style. Preferred agents according to the invention are therefore characterized in that they have a pH value of <5, preferably <4, more preferably of 2.5 to 3.5 and in particular of 2.7 to 3.3.

As a further optional constituent, the agents according to the invention can include 0.01 to 10 wt. % of at least one polymer from the group of the cationic and/or amphoteric polymers.

The cationic polymers can be homo- or copolymers, wherein the quaternary nitrogen groups are included either in the polymer chain or preferably as a substituent on one or more of the monomers. The monomers that include ammonium groups can be copolymerized with non-cationic monomers. Suitable cationic monomers are unsaturated compounds capable of free-radical polymerization, which carry at least one cationic group, in particular ammonium-substituted vinyl monomers, such as e.g. trialkyl-methacryloxyalkylammonium, trialkylacryloxyalkylammonium, dialkyldiallylammonium and quaternary vinylammonium monomers with cyclic groups that include cationic nitrogens, such as pyridinium, imidazolium or quaternary pyiTolidones, e.g. alkylvinylimidazolium, alkylvinylpyridinium or alkylvinylpyrrolidone salts. The alkyl groups of these monomers are preferably lower alkyl groups, such as e.g. C1 to C7 alkyl groups, more preferably C1 to C3 alkyl groups.

The monomers that include ammonium groups can be copolymerized with non-cationic monomers. Suitable comonomers are e.g. acrylamide, methacrylamide; alkyl and dialkyl acrylamide, alkyl and dialkyl methacrylamide, alkyl acrylate, alkyl methacrylate, vinyl caprolactone, vinylcaprolactam, vinylpyrrolidone, vinyl esters, e.g. vinyl acetate, vinyl alcohol, propylene glycol or ethylene glycol, the alkyl groups of these monomers preferably being C1 to C7 alkyl groups and more preferably C1 to C3 alkyl groups.

Suitable polymers with quaternary amine groups are e.g. the polymers described in the CTFA Cosmetic Ingredient Dictionary with the names polyquarternium, such as methylvinylimidazolium chloride/vinylpyrrolidone copolymer (polyquarternium-16) or quaternized vinylpyrrolidone/dimethylamino ethyl methacrylate copolymer (polyquarternium-11).

Of the cationic polymers that can be included in the agent according to the invention, for example vinylpyrrolidone/dimethylaminoethyl methacrylate methosulfate copolymer, which is marketed by Gaf Co., USA with the trade names Gafquat® 755 N and Gafquat® 734, of which Gafquat® 734 is more preferred, are suitable. Other cationic polymers are e.g. the copolymer of polyvinylpyrrolidone and imidazolimine methochloride marketed by BASF, Germany with the trade name Luviquat® HM 550, the terpolymer of dimethyldiallylammonium chloride, sodium acrylate and acrylamide marketed by Calgon, USA with the trade name Merquat® Plus 3300 and the vinylpyrrolidone/methacrylamidopropyltrimethylammonium chloride copolymer marketed by ISP with the trade name Gafquat® HS 100.

Homopolymers of the general formula (P1), —{CH₂-[CR¹COO—(CH₂)ₘN⁺R²R³R⁴]}ₙ X⁻,

in which R¹=—H or —CH₃, R², R³ and R⁴ independently of one another are selected from C1-4 alkyl, alkenyl or hydroxyalkyl groups, m=1, 2, 3 or 4, n is a natural number and
X⁻ is a physiologically acceptable organic or inorganic anion. In the context of these polymers, those for which at least one of the following conditions applies are preferred according to the invention: R¹ denotes a methyl group, R², R³ and R⁴ denote methyl groups, m has the value 2.

As physiologically acceptable counter-ions X⁻, e.g. halide ions, sulfate ions, phosphate ions, methosulfate ions as well as organic ions such as lactate, citrate, tartrate and acetate ions are suitable. Halide ions are preferred, in particular chloride.

A more suitable homopolymer is the optionally crosslinked poly(methacryloyloxyethyltrimethylammonium chloride) with the INCI name polyquarternium-37. Products of this type are commercially available e.g. with the names Rheocare® CTH (Cosmetic Rheologies) and Synthalen® CR (3V Sigma).

The homopolymer is preferably employed in the form of a non-aqueous polymer dispersion. Polymer dispersions of this type are commercially available with the names Salcare® SC 95 and Salcare® SC 96.

A preferred copolymer according to the invention is the crosslinked acrylamide-methacryloyloxyethyltrimethylammonium chloride copolymer. Copolymers of this type are commercially available with the name Salcare® SC 92.

Suitable cationic polymers that are derived from natural polymers are cationic derivatives of polysaccharides, e.g. cationic derivatives of cellulose, starch or guar. Also suitable are chitosan and chitosan derivatives. Cationic polysaccharides have the general formula (P-3) G-O—B—N+RₐR_{b}R_{c}X⁻

G is an anhydroglucose residue, e.g. starch or cellulose anhydroglucose;
B is a group of divalent compounds, e.g. alkylene, oxyalkylene, polyoxyalkylene or hydroxyalkylene;
Rₐ, R_{b} and R_{c}, independently of one another, are alkyl, aryl, alkylaryl, arylalkyl, alkoxyalkyl or alkoxyaryl having up to 18 C atoms each, wherein the total number of C atoms in Rₐ, R_{b} and R_{c} is preferably no more than 20;
X⁻ is a conventional counter-anion and is preferably chloride.

A cationic cellulose is marketed by Amerchol with the name Polymer JR® 400 and has the INCI name polyquarternium-10. Another cationic cellulose has the INCI name polyquarternium-24 and is marketed by Amerchol with the trade name Polymer LM-200. Other commercial products are the compounds Celquat® H 100, Celquat® L 200. The above mentioned commercial products are preferred cationic celluloses. Other preferred cationic celluloses are known by the INCI names polyquarternium-67 and polyquarternium-72.

Suitable cationic guar derivatives are marketed with the trade name Jaguar® and have the INCI name guar hydroxypropyltrimonium chloride. Particularly suitable cationic guar derivatives are also commercially available from Hercules with the name N-Hance®. Other cationic guar derivatives are marketed by Cognis with the name Cosmedia®. A preferred cationic guar derivative is the commercial product AquaCat® from Hercules. This raw material is a pre-dissolved cationic guar derivative.

A suitable chitosan is marketed e.g. by Kyowa Oil & Fat, Japan, with the trade name Flonac®. A preferred chitosan salt is chitosonium pyrrolidone carboxylate, which is marketed e.g. by Amerchol, USA with the name Kytamer° PC. Other chitosan derivatives are readily available on the market with the trade names Hydagen® CMF, Hydagen® HCMF and Chitolam® NB/101.

Other preferred cationic polymers are e.g.
a. cationic alkyl polyglycosides,
b. cationized honey, e.g. the commercial product Honeyquat® 50,
c. polymeric dimethyldiallylammonium salts and copolymers thereof with esters and amides of acrylic acid and methacrylic acid. The products that are commercially available with the names Merquat® 100 (poly(dimethyldiallylammonium chloride)) and Merquat® 550 (dimethyldiallylammonium chloride-acrylamide copolymer) are examples of these cationic polymers,
d. vinylpyrrolidone-vinylimidazolium methochloride copolymers, as offered with the names Luviquat® FC 370, FC 550, FC 905 and HM 552,
e. quaternized polyvinyl alcohol,
f. as well as the polymers known by the names polyquarternium-2, polyquarternium-17, polyquarternium-18 and polyquarternium-27 with quaternary nitrogen atoms in the polymer main chain,
g. vinylpyrrolidone-vinylcaprolactam-acrylate terpolymers, as offered for sale with acrylic acid esters and acrylic acid amides as the third monomer building block, e.g. with the name Aquaflex® SF 40.

It is likewise possible according to the invention to use the copolymers of vinylpyrrolidone as available as the commercial products Copolymer 845 (manufacturer: ISP), Gaffix® VC 713 (manufacturer: ISP), Gafquat® ASCP 1011, Gafquat® HS 110, Luviquat® 8155 and Luviquat® MS 370.

The cationic polymers are included in the compositions according to the invention preferably in quantities of 0.01 to 10 wt. %, based on the total agent. Quantities of 0.05 to 5 wt. % are more preferred.

Regardless of whether or not amphoteric polymers are included in the agents, further preferred agents according to the invention are characterized in that they include—based on their weight—0.05 to 7.5 wt. %, preferably 0.1 to 5 wt. %, more preferably 0.2 to 3.5 wt. % and in particular 0.25 to 2.5 wt. % cationic polymer(s).

In summary, agents according to the invention are preferred which include—based on their weight—0.05 to 7.5 wt. %, preferably 0.1 to 5 wt. %, more preferably 0.2 to 3.5 wt. % and in particular 0.25 to 2.5 wt. % cationic polymer(s), wherein preferred cationic polymer(s) is/are selected from
a. poly(methacryloyloxyethyltrimethylammonium chloride) (INCI: polyquarternium-37) and/or;
b. quaternized cellulose derivatives (INCI: polyquarternium 10) and/or
c. cationic alkyl polyglycosides and/or
d. cationized honey and/or
e. cationic guar derivatives and/or
f. polymeric dimethyldiallylammonium salts and copolymers thereof with esters and amides of acrylic acid and methacrylic acid and/or
g. copolymers of vinylpyrrolidone with quaternized derivatives of dialkyl aminoalkyl acrylate and methacrylate and/or
h. vinylpyrrolidone-vinylimidazolium methochloride copolymers and/or
i. quaternized polyvinyl alcohol and/or
j. polyquarternium 2 and/or
k. polyquarternium-7 and/or
l. polyquarternium-16 and/or
m. polyquarternium 17 and/or
n. polyquarternium 18 and/or
o. polyquarternium 24 and/or
p. polyquarternium 27.

Furthermore, amphoteric polymers can be used as polymers. The term amphoteric polymers covers those polymers that include both free amino groups and free —COOH or SO₃H groups in the molecule and are capable of forming inner salts, as well as zwitterionic polymers which include quaternary ammonium groups and —COO⁻ or —SO₃⁻ groups in the molecule, and those polymers which include —COOH or SO₃H groups and quaternary ammonium groups.

Preferred amphoteric and/or cationic polymers according to the invention are those polymers in which a cationic group is derived from at least one of the following monomers:

(i) monomers with quaternary ammonium groups of the general formula (Mono1),

R¹—CH═CR²—CO—Z—(CₙH₂ₙ)—N⁽⁺⁾R²R³R⁴A⁽⁻⁾ (Mono1)

in which R¹ and R² independently of one another denote hydrogen or a methyl group and R³, R⁴ and R⁵ independently of one another denote alkyl groups with 1 to 4 carbon atoms, Z is an NH group or an oxygen atom, n is an integer from 2 to 5 and A⁽⁻⁾ is the anion of an organic or inorganic acid,
(ii) monomers with quaternary ammonium groups of the general formula (Mono2),

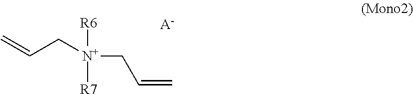

where R⁶ and R⁷ independently of one another denote a (C₁ to C4) alkyl group, in particular a methyl group, and
A⁻ is the anion of an organic or inorganic acid,
(iii) monomeric carboxylic acids of the general formula (Mono3),

R⁸—CH═CR⁹—COOH (Mono3)

in which R⁸ and R⁹ independently of one another are hydrogen or methyl groups.

More preferred are those polymers in which monomers of type (i) are employed, in which R³, R⁴ and R⁵ are methyl groups, Z is an NH group and A⁽⁻⁾ is a halide, methoxysulfate or ethoxysulfate ion; acrylamidopropyltrimethylammonium chloride is a particularly preferred monomer (i). As monomer (ii) for the above-mentioned polymers, acrylic acid is preferably used.

More preferred amphoteric polymers are copolymers of at least one monomer (Mono1) or (Mono2) with the monomer (Mono3), in particular copolymers of the monomers (Mono2) and (Mono3). Particularly preferably used amphoteric polymers according to the invention are copolymers of diallyldimethylammonium chloride and acrylic acid. These copolymers are marketed with the INCI name polyquarternium-22, inter alia with the trade name Merquat® 280 (Nalco).

In addition, besides a monomer (Mono1) or (Mono2) and a monomer (Mono3), the amphoteric polymers according to the invention can additionally include a monomer (Mono4)

(iv) monomeric carboxylic acid amides of the general formula (Mono4),

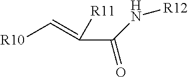

in which R¹⁰ and R¹¹ independently of one another are hydrogen or methyl groups and R¹² denotes a hydrogen atom or a (C₁ to C₈) alkyl group.

Particularly preferably used amphoteric polymers according to the invention based on a comonomer (Mono4) are terpolymers of diallyldimethylammonium chloride, acrylamide and acrylic acid. These copolymers are marketed with the INCI name polyquarternium-39, inter alia with the trade name Merquat® Plus 3330 (Nalco).

The amphoteric polymers can generally be employed according to the invention either directly or in the form of a salt, which is obtained by neutralization of the polymers, e.g. with an alkali hydroxide.

With particular preference, the amphoteric polymers employed in the agents according to the invention include monomers from the group of the acrylamides and/or methacrylamides with alkylammonium groups. Acrylic acid and/or methacrylic acid and/or crotonic acid and/or 2-methylcrotonic acid have proved suitable as monomers with anionic groups that are additionally included in the polymers.

The amphoteric polymer or polymers is/are preferably employed within relatively narrow quantitative ranges. Thus, agents according to the invention are preferred which include—based on their weight—0.05 to 7.5 wt. %, preferably 0.1 to 5 wt. %, more preferably 0.2 to 3.5 wt. % and in particular 0.25 to 2.5 wt. % amphoteric polymer(s).

The anionic polymers are anionic polymers that have carboxylate and/or sulfonate groups. Examples of anionic monomers of which these polymers can consist are acrylic acid, methacrylic acid, crotonic acid, maleic anhydride and 2-acrylamido-2-methylpropanesulfonic acid. The acid groups here can be completely or partially present as a sodium, potassium, ammonium, mono- or triethanolammonium salt. Preferred monomers are 2-acrylamido-2-methylpropanesulfonic acid and acrylic acid.

Anionic polymers that include 2-acrylamido-2-methylpropanesulfonic acid as the sole monomer or as a comonomer, wherein the sulfonic acid group can be completely or partially present as a sodium, potassium, ammonium, mono- or triethanolammonium salt, have proved particularly effective.

The homopolymer of 2-acrylamido-2-methylpropanesulfonic acid, which is commercially available e.g. with the name Rheothik 11-80, is more preferred.

Within this embodiment it may be preferred to employ copolymers of at least one anionic monomer and at least one non-ionogenic monomer. With regard to the anionic monomers, reference is made to the substances mentioned above. Preferred non-ionogenic monomers are acrylamide, methacrylamide, acrylic acid esters, methacrylic acid esters, vinylpyrrolidone, vinyl ethers and vinyl esters.

Preferred anionic copolymers are acrylic acid-acrylamide copolymers as well as in particular polyacrylamide copolymers with monomers that include sulfonic acid groups. A polymer of this type is included in the commercial product Sepigel® 305 from SEPPIC.

The sodium acryloyl dimethyl taurate copolymers marketed with the name Simulgel® 600 as a compound with isohexadecane and polysorbate-80 have also proved particularly effective according to the invention.

Uncrosslinked and crosslinked polyacrylic acids are likewise preferred anionic homopolymers. Allyl ethers of pentaerythritol, of sucrose and of propylene can be preferred crosslinking agents here. Compounds of this type are commercially available e.g. with the trade mark Carbopol®.

Copolymers of maleic anhydride and methyl vinyl ether, in particular those with crosslinkages, are likewise color-retaining polymers. A maleic acid-methyl vinyl ether copolymer crosslinked with 1,9-decadiene is commercially available with the name Stabileze® QM.

The anionic polymers are included in the agents according to the invention preferably in quantities of 0.05 to 10 wt. %, based on the total agent. Quantities of 0.1 to 5 wt. % are more preferred.

A particularly preferred polyurethane according to the invention is on the market with the trade name Luviset® PUR (BASF).

In another embodiment, the agents according to the invention can include non-ionogenic polymers.

Suitable non-ionogenic polymers are e.g.
vinylpyrrolidone/vinyl ester copolymers, as marketed e.g. with the trademark Luviskol® (BASF). Luviskol® VA 64 and Luviskol® VA 73, both of them vinylpyrrolidone/vinyl acetate copolymers, are likewise preferred nonionic polymers;
cellulose ethers, such as hydroxypropyl cellulose, hydroxyethyl cellulose and methyl hydroxypropyl cellulose, as marketed e.g. with the trademarks Culniinal® and Benecel® (AQUALON) and Natrosol® grades (Hercules);
starch and derivatives thereof, in particular starch ethers, e.g. Structure® XL (National Starch), a multifunctional, salt-tolerant starch;
shellac;
polyvinylpyrrolidones, as marketed e.g. with the name Luviskol® (BASF).

The nonionic polymers are included in the compositions according to the invention preferably in quantities of 0.05 to 10 wt. %, based on the total agent. Quantities of 0.1 to 5 wt. % are more preferred.

It is also possible according to the invention that the preparations used include multiple, in particular two different polymers with the same charge and/or in each case an ionic and an amphoteric and/or non-ionic polymer.

In another preferred embodiment of the invention, the action of the active agent according to the invention can be further optimized by fatty substances. Fatty substances are to be understood as fatty acids, fatty alcohols, natural and synthetic waxes, which can be present both in solid form and as a liquid in aqueous dispersion, and natural and synthetic cosmetic oil components.

As fatty acids it is possible to employ linear and/or branched, saturated and/or unsaturated fatty acids with 6-30 carbon atoms. Fatty acids with 10-22 carbon atoms are preferred. Among these, e.g. the isostearic acids, such as the commercial products Emersol° 871 and Emersol® 875, and isopalmitic acids, such as the commercial product Edenor® IP 95, should be mentioned as well as all other fatty acids marketed with the trade names Edenor® (Cognis). Further typical examples of these fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidic acid, gadoleic acid, behenic acid and erucic acid as well as technical mixtures thereof which are obtained e.g. by hydrolysis of natural fats and oils under pressure, oxidation of aldehydes from Roelen's oxo synthesis or dimerization of unsaturated fatty acids. More preferred generally are the fatty acid blends that are obtainable from coconut oil or palm oil; the use of stearic acid is usually particularly preferred.

The quantity employed in this case is 0.1-15 wt. %, based on the total agent. In a preferred embodiment, the quantity is 0.5-10 wt. %, with quantities of 1-5 wt. % being particularly advantageous.

As fatty alcohols, it is possible to employ saturated, mono- or polyunsaturated, branched or unbranched fatty alcohols with C₆-C₃₀, preferably C₁₀-C₂₂ and particularly preferably C₁₂-C₂₂ carbon atoms. For example, decanol, octanol, octenol, dodecenol, decenol, octadienol, dodecadienol, decadienol, oleyl alcohol, erucyl alcohol, ricinoleyl alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol and the Guerbet alcohols thereof can be employed within the meaning of the invention, this list being intended to be of an exemplary and non-limiting nature. However, the fatty alcohols are derived from preferably natural fatty acids, wherein it can generally be assumed that they are obtained from the esters of the fatty acids by reduction. According to the invention, it is likewise possible to employ those fatty alcohol blends that are produced by reduction of naturally occurring triglycerides, such as beef tallow, palm oil, ground nut oil, rapeseed oil, cottonseed oil, soybean oil, sunflower oil and linseed oil or fatty acid esters formed from the transesterification products thereof with corresponding alcohols, and which thus represent a mixture of different fatty alcohols. Substances of this type can be purchased e.g. with the names Stenol®, e.g. Stenol® 1618, or Lanette®, e.g. Lanette® O, or Lorol®, e.g. Lorol® C8, Lorol® C14, Lorol® C18, Lorol® C8-18, HD-Ocenol®, Crodacol, e.g. Crodacol® CS, Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. It is, of course, also possible according to the invention to employ wool wax alcohols, as can be purchased e.g. with the names Corona®, White Swan®, Coronet® or Fluilan®. The fatty alcohols are employed in quantities of 0.1-10 wt. %, based on the total preparation, preferably in quantities of 0.1-5.0 wt. %.

As natural or synthetic waxes, it is possible according to the invention to employ solid paraffins or isoparaffins, carnauba waxes, beeswaxes, candelilla waxes, ozokerites, ceresin, cetaceum, sunflower wax, fruit waxes, such as e.g. apple wax or citrus wax, micro waxes comprising PE or PP. Waxes of this type are available e.g. through Kahl & Co., Trittau.

Furthermore, it has been demonstrated that the action of the active agent according to the invention can be enhanced if it is combined with hydroxycarboxylic acid esters. Preferred hydroxycarboxylic acid esters are full esters of glycolic acid, lactic acid, malic acid, tartaric acid or citric acid. Other hydroxycarboxylic acid esters that are suitable in principle are esters of β-hydroxypropionic acid, tartronic acid, D-gluconic acid, sugar acid, mucic acid or glucuronic acid. As the alcohol component of these esters, primary, linear or branched aliphatic alcohols with 8-22 C atoms, i.e. for example fatty alcohols or synthetic fatty alcohols, are suitable. The esters of C12-C15 fatty alcohols are more preferred here. Esters of this type are commercially available, e.g. with the trade mark Cosmacol® from EniChem, Augusta Industriale. The quantity of the hydroxycarboxylic acid esters employed in this case is 0.1-15 wt. % based on the agent, preferably 0.1-10 wt. % and particularly preferably 0.1-5 wt. %.

As a further ingredient, the agents according to the invention can, with particular preference, include one or more amino acids Amino acids that can be more preferably used according to the invention come from the group of glycine, alanine, valine, leucine, isoleucine, phenylalanine, tyrosine, tryptophan, proline, aspartic acid, glutamic acid, asparagine, glutamine, serine, threonine, cysteine, methionine, lysine, arginine, histidine, β-alanine, 4-aminobutyric acid (GABA), betaine, L-cystine (L-Cys), L-carnitine, L-citrulline, L-theanine, 3′,4′-dihydroxy-L-phenylalanine (L-Dopa), 5′-hydroxy-L-tryptophan, L-homocysteine, S-methyl-L-methionine, S-allyl-L-cysteine sulfoxide (L-alliin), L-trans-4-hydroxyproline, L-5-oxoproline (L-pyroglutamic acid), L-phosphoserine, creatine, 3-methyl-L-histidine and L-ornithine, it being possible to use both the individual amino acids and mixtures.

Preferred agents according to the invention include one or more amino acids in relatively narrow quantitative ranges. In this case, preferred hair treatment agents according to the invention are characterized in that they include as care substance—based on their weight—0.01 to 5 wt. %, preferably 0.02 to 2.5 wt. %, more preferably 0.05 to 1.5 wt. %, still more preferably 0.075 to 1 wt. % and in particular 0.1 to 0.25 wt. % amino acid(s), preferably from the group of glycine and/or alanine and/or valine and/or lysine and/or leucine and/or threonine.

Another more preferred group of ingredients of the agents according to the invention are vitamins, provitamins or vitamin precursors. These are described below:

The group of substances referred to as vitamin A includes retinol (vitamin A₁) and 3,4-didehydroretinol (vitamin A₂). β-Carotene is the provitamin of retinol. Suitable according to the invention as vitamin A component are e.g. vitamin A acid and esters thereof, vitamin A aldehyde and vitamin A alcohol and esters thereof, such as the palmitate and acetate. The agents according to the invention include the vitamin A component preferably in quantities of 0.05-1 wt. %, based on the total preparation.

The vitamin B group or vitamin B complex includes, inter alia:
Vitamin B₁ (thiamine)
Vitamin B₂ (riboflavin)
Vitamin B₃. This name often covers the compounds nicotinic acid and nicotinamide (niacinamide). Preferred according to the invention is nicotinamide, which is included in the agents used according to the invention preferably in quantities of 0.05 to 1 wt. %, based on the total agent.
Vitamin B₅ (pantothenic acid, panthenol and pantolactone). Within the context of this group, preferably panthenol and/or pantolactone is employed. Derivatives of panthenol that can be employed according to the invention are in particular the esters and ethers of panthenol and cationically derivatized panthenols. Individual representatives are e.g. panthenol triacetate, panthenol monoethyl ether and monoacetate thereof as well as the cationic panthenol derivatives. The above-mentioned compounds of the vitamin B₅ type are included in the agents according to the invention preferably in quantities of 0.05-10 wt. %, based on the total agent. Quantities of 0.1-5 wt. % are more preferred.
Vitamin B₆ (pyridoxine as well as pyridoxamine and pyridoxal).

Vitamin C (ascorbic acid). Vitamin C is employed in the agents according to the invention preferably in quantities of 0.1 to 3 wt. %, based on the total agent. Use in the form of the palmitic acid ester, glucosides or phosphates can be preferred. Use in combination with tocopherols can likewise be preferred.

Vitamin E (tocopherols, in particular α-tocopherol). Tocopherol and derivatives thereof, including in particular the esters, such as the acetate, the nicotinate, the phosphate and the succinate, are included in the agents according to the invention preferably in quantities of 0.05-1 wt. %, based on the total agent.

Vitamin F. The term “vitamin F” is generally understood to mean essential fatty acids, in particular linoleic acid, linolenic acid and arachidonic acid.

Vitamin H. The compound (3aS,4S,6aR)-2-oxohexahydrothieno[3,4-d]-imidazole-4-valeric acid is referred to as vitamin H, but its trivial name biotin has now become accepted. Biotin is included in the agents according to the invention preferably in quantities of 0.0001 to 1.0 wt. %, in particular in quantities of 0.001 to 0.01 wt. %.

In summary, hair treatment agents according to the invention are preferred which additionally include as care substance—based on its weight—0.1 to 5 wt. %, preferably 0.2 to 4 wt. %, more preferably 0.25 to 3.5 wt. %, still more preferably 0.5 to 3 wt. % and in particular 0.5 to 2.5 wt. % vitamins and/or pro-vitamins and/or vitamin precursors which are preferably assigned to the groups A, B, C, E, F and H, wherein preferred agents include panthenol ((±)-2,4-dihydroxy-N-(3-hydroxypropyl)-3,3-dimethylbutyramide, provitamin B₅) and/or pantothenic acid (vitamin B₃, vitamin B₅) and/or niacin, niacinamide or nicotinamide (vitamin B₃) and/or L-ascorbic acid (vitamin C) and/or thiamine (vitamin B₁) and/or riboflavin (vitamin B₂, vitamin G) and/or biotin (vitamin B₇, vitamin H) and/or folic acid (vitamin B₉, vitamin B_{c} or vitamin M) and/or vitamin B₆ and/or vitamin B₁₂.

It has been shown that the use of specific quinones reinforces an anti-dandruff and anti-hair loss action as well as bringing about advantages in terms of combability and gloss. As a further constituent, the agents according to the invention can therefore include 0.0001 to 5 wt. % of at least one bioquinone and/or plastoquinone. The preferred ubiquinones according to the invention have the following formula:

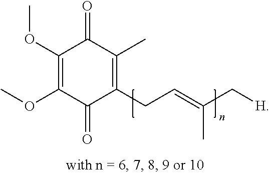

Coenzyme Q-10 is most preferred here.

To improve the elasticity and strengthen the internal structure of hair treated with agents according to the invention, the agents according to the invention can include purine and/or purine derivatives. In particular the combination of purine and/or purine derivatives with ubiquinones and/or plastoquinones leads inter alia to higher measured values in differential thermal analysis and improved wet and dry combability in hair treated with corresponding agents.

Preferred compositions according to the invention include purine and/or purine derivatives in relatively narrow quantitative ranges. In this case, preferred cosmetic agents according to the invention are characterized in that they include—based on their weight—0.001 to 2.5 wt. %, preferably 0.0025 to 1 wt. %, more preferably 0.005 to 0.5 wt. % and in particular 0.01 to 0.1 wt. % purine(s) and/or purine derivative(s). Preferred cosmetic agents according to the invention are characterized in that they include purine, adenine, guanine, uric acid, hypoxanthine, 6-purinethiol, 6-thioguanine, xanthine, caffeine, theobromine or theophylline. Caffeine is most preferred in hair cosmetic preparations.

It is furthermore advantageous to use purine or purine derivatives and bioquinones in a specific ratio to one another. In this case, agents according to the invention are preferred in which the weight ratio of ingredients a) and b) is 10:1 to 1:100, preferably 5:1 to 1:50, more preferably 2:1 to 1:20 and in particular 1:1 to 1:10.

As already mentioned, caffeine is a more preferred purine derivative and coenzyme Q10 is a more preferred bioquinone. More preferred agents according to the invention are therefore characterized in that they include—based on their weight—0.001 to 2.5 wt. %, preferably 0.0025 to 1 wt. %, more preferably 0.005 to 0.5 wt. % and in particular 0.01 to 0.1 wt. % caffeine and 0.0002 to 4 wt. %, preferably 0.0005 to 3 wt. %, more preferably 0.001 to 2 wt. %, still more preferably 0.0015 to 1 and in particular 0.002 to 0.5 wt. % coenzyme Q10.

As a further constituent, the agents according to the invention can include at least one carbohydrate from the group of the monosaccharides, disaccharides and/or oligosaccharides. In this case, preferred hair treatment agents according to the invention are characterized in that they include as care substance—based on their weight—0.01 to 5 wt. %, preferably 0.05 to 4.5 wt. %, more preferably 0.1 to 4 wt. %, still more preferably 0.5 to 3.5 wt. % and in particular 0.75 to 2.5 wt. % carbohydrate(s), selected from monosaccharides, disaccharides and/or oligosaccharides, wherein preferred carbohydrates are selected from
monosaccharides, in particular D-ribose and/or D-xylose and/or L-arabinose and/or D-glucose and/or D-mannose and/or D-galactose and/or D-fructose and/or sorbose and/or L-fucose and/or L-rhamnose
disaccharides, in particular sucrose and/or maltose and/or lactose and/or trehalose and/or cellobiose and/or gentiobiose and/or isomaltose.

As already mentioned, preferred agents according to the invention include (an) amino acid(s).

Amino acids that can be more preferably employed according to the invention come from the group of glycine, alanine, valine, leucine, isoleucine, phenylalanine, tyrosine, tryptophan, proline, aspartic acid, glutamic acid, asparagine, glutamine, serine, threonine, cysteine, methionine, lysine, arginine, histidine, β-alanine, 4-aminobutyric acid (GABA), betaine, L-cystine (L-Cyss), L-carnitine, L-citrulline, L-theanine, 3′,4′-dihydroxy-L-phenylalanine (L-Dopa), 5′-hydroxy-L-tryptophan, L-homocysteine, S-methyl-L-methionine, S-allyl-L-cysteine sulfoxide (L-alliin), L-trans-4-hydroxyproline, L-5-oxoproline (L-pyroglutamic acid), L-phosphoserine, creatine, 3-methyl-L-histidine and L-ornithine, it being possible to employ both the individual amino acids and mixtures.

Preferred agents according to the invention include one or more amino acids in relatively narrow quantitative ranges. In this case, preferred cosmetic agents according to the invention are characterized in that they additionally include 0.05 to 5 wt. %, preferably 0.1 to 2.5 wt. %, more preferably 0.15 to 1 wt. % and in particular 0.2 to 0.5 wt. % amino acid(s), preferably (an) amino acid(s) from the group of glycine and/or alanine and/or valine and/or lysine and/or leucine and/or threonine.

Another preferred care substance that can be employed, which possesses activating properties, is taurine. Preferred hair treatment agents according to the invention include as care substance—based on their weight—0.01 to 15 wt. %, preferably 0.025 to 12.5 wt. %, more preferably 0.05 to 10 wt. %, still more preferably 0.1 to 7.5 wt. % and in particular 0.5 to 5 wt. % taurine (2-aminoethanesulfonic acid).

Also preferred is the additional use of bisabolol and/or bisabolol oxides in the agents according to the invention. In this case, hair treatment agents according to the invention are preferred which additionally include 0.001 to 5 wt. %, preferably 0.01 to 4 wt. %, more preferably 0.02 to 2.5 wt. % and in particular 0.1 to 1.5 wt. % bisabolol and/or oxides of bisabolol, preferably (−)-alpha-bisabolol

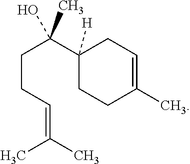 [SMILES: CC(C)=CCC[C@](C)(O)[C@H]1CC=C(C)CC1]

Furthermore, the agents according to the invention should additionally include at least one UV light protective filter. UVB filters can be oil-soluble or water-soluble.

As oil-soluble substances, e.g. the following should be mentioned:
3-benzylidene camphor, e.g. 3-(4-methylbenzylidene) camphor;
4-aminobenzoic acid derivatives, preferably 4-(dimethylamino)benzoic acid 2-ethylhexyl ester, 4-(dimethylamino)benzoic acid 2-octyl ester and 4-(dimethylamino)benzoic acid amyl ester;
esters of cinnamic acid, preferably 4-methoxycinnamic acid 2-ethylhexyl ester, 4-methoxycinnamic acid propyl ester, 4-methoxycinnamic acid isoamyl ester, 2-cyano-3-phenylcinnamic acid 2-ethylhexyl ester (octocrylene);
esters of salicylic acid, preferably salicylic acid 2-ethylhexyl ester, salicylic acid 4-isopropylbenzyl ester, salicylic acid homomenthyl ester;
derivatives of benzophenone, preferably 2-hydroxy-4-methoxybenzophenone, 2-hydroxy-4-methoxy-4′-methylbenzophenone, 2,2′-dihydroxy-4-methoxybenzophenone;
esters of benzalmalonic acid, preferably 4-methoxybenzmalonic acid di-2-ethylhexyl ester;
triazine derivatives, such as e.g. 2,4,6-trianilino-(p-carbo-2′-ethyl-1′-hexyloxy)-1,3,5-triazine and octyl triazone;
propane-1,3-diones, such as e.g. 1-(4-tert.-butylphenyl)-3-(4′-methoxyphenyl)propane-1,3-dione.

The following are suitable as water-soluble substances:
2-phenylbenzimidazole-5-sulfonic acid and alkali, alkaline earth, ammonium, alkylammonium, alkanolammonium and glucammonium salts thereof;
sulfonic acid derivatives of benzophenones, preferably 2-hydroxy-4-methoxybenzophenone-5-sulfonic acid and salts thereof;
sulfonic acid derivatives of 3-benzylidene camphor, such as e.g. 4-(2-oxo-3-bornylidenemethyl)benzenesulfonic acid and 2-methyl-5-(2-oxo-3-bomylidene)sulfonic acid and salts thereof.

As typical UV-A filters, derivatives of benzoyl methane are particularly suitable, such as e.g. 1-(4′-tert.-butylphenyl)-3-(4′-methoxyphenyl)propane-1,3-dione or 1-phenyl-3-(4′-isopropylphenyl)propane-1,3-dione. The UV-A and UV-B filters can, of course, also be used in mixtures. Besides the aforementioned soluble substances, insoluble pigments are also suitable for this purpose, in particular finely dispersed metal oxides or salts, such as e.g. titanium dioxide, zinc oxide, iron oxide, aluminum oxide, cerium oxide, zirconium oxide, silicates (talc), barium sulfate and zinc stearate. The particles in this case should have an average diameter of less than 100 nm, preferably between 5 and 50 nm and in particular between 15 and 30 nm. They can have a spherical shape, but those particles that possess an ellipsoid shape or a shape deviating from the spherical form in another way can also be employed.

The UV filters are generally included in the agents according to the invention in quantities of 0.1-5 wt. %, based on the total agent. Quantities of 0.4-2.5 wt. % are preferred.

The agents according to the invention can furthermore include a 2-pyrrolidinone-5-carboxylic acid and derivatives thereof. Preferred are the sodium, potassium, calcium, magnesium or ammonium salts in which the ammonium ion carries one to three C₁ to C₄ alkyl groups besides hydrogen. The sodium salt is particularly preferred. The quantities employed in the agents according to the invention are preferably 0.05 to 10 wt. %, based on the total agent, more preferably 0.1 to 5 and in particular 0.1 to 3 wt. %.

Finally, the agents according to the invention can also include plant extracts.

These extracts are generally produced by extraction of the whole plant. In individual cases, however, it may also be preferred to produce the extracts exclusively from flowers and/or leaves of the plant.

With regard to the plant extracts that can be used according to the invention, particular reference is made to the extracts listed in the table starting on page 44 of the 3rd edition of the Leitfaden zur Inhaltsstoffdeklaration kosmetischer Mittel, published by the Industrieverband Körperpflege- and Waschmittel e.V. (IKW), Frankfurt.

According to the invention, primarily the extracts of green tea, oak bark, nettles, hamamelis, hops, henna, chamomile, burdock root, horsetail, hawthorn, lime blossom, almond, aloe vera, fir needle, horse chestnut, sandalwood, juniper, coconut, mango, apricot, lime, wheat, kiwi, melon, orange, grapefruit, sage, rosemary, birch, mallow, lady's smock, wild thyme, yarrow, thyme, melissa, rest harrow, coltsfoot, marsh mallow, meristem, ginseng and ginger root are preferred.

More preferred are the extracts of green tea, oak bark, nettles, hamamelis, hops, chamomile, burdock root, horsetail, lime blossom, almond, aloe vera, coconut, mango, apricot, lime, wheat, kiwi, melon, orange, grapefruit, sage, rosemary, birch, lady's smock, wild thyme, yarrow, rest harrow, meristem, ginseng and ginger root.

Particularly suitable for the use according to the invention are the extracts of green tea, almond, aloe vera, coconut, mango, apricot, lime, wheat, kiwi and melon.

The plant extracts can be employed according to the invention both in pure form and in dilute form. Where they are employed in dilute form, they usually include approx. 2-80 wt. % active substance and, as solvent, the extracting agent or mixture of extracting agents employed to obtain them.

Furthermore, it can be preferred to employ mixtures of multiple, in particular of two, different plant extracts in the agents according to the invention.

In addition, it may prove advantageous if penetration auxiliaries and/or swelling agents are included in the agents according to the invention. These can include, for example, urea and urea derivatives, guanidine and derivatives thereof, arginine and derivatives thereof, water glass, imidazole and derivatives thereof, histidine and derivatives thereof, benzyl alcohol, carbonates, hydrogen carbonates.

The agents according to the invention exhibit advantageous properties and also impart advantageous properties to hair treated therewith. Advantages were observed in hair conditioning in particular. For instance, hair treatment agents according to the invention improve the handle and combability in the dry and wet state as well as the gloss of hair treated therewith. A prevention of premature split ends is also shown in treated hair without any negative effect on volume and fullness.

The present invention therefore also provides a method for human haircare, in which an agent according to the invention is applied onto the hair, left on the hair for a period of exposure of 10 to 600 seconds, preferably of 30 to 150 seconds, and then rinsed out of the hair.

The present invention also provides a method for human haircare, in which an agent according to the invention is applied onto the hair and remains there until the next hair wash, i.e. is not rinsed out again after a period of exposure of a few seconds. This method is preferred according to the invention.

With regard to further preferred embodiments of the methods according to the invention, the statements made relating to the agents according to the invention apply mutatis mutandis.

The present invention also provides the use of agents according to the invention for non-weighing haircare, in particular for reducing combing forces in wet and dry hair, for increasing gloss and for reducing split ends.

The compositions according to the invention can be formulated both as an aerosol and as a non-aerosol. As a non-aerosol, the compositions according to the invention are applied onto the hair from containers which are known per se to the person skilled in the art and which are preferably of clear and transparent design, with the aid of a conventional spray pump device. However, the application can, of course, also take place simply by pouring. In each case, the three phases have to be briefly homogenized by shaking beforehand.

### EXAMPLES

All quantitative data are parts by weight, unless otherwise specified. The following formulations were prepared using known production methods.

| | 1 | 2 | 3 | 4 | 5 | 6 | 7 | 8 |
|---|---|---|---|---|---|---|---|---|
| Water | 80.0 | 80.0 | 75.0 | 80.0 | 50.0 | 50.0 | 85.0 | 85.0 |
| Ethanol | — | — | — | — | 40.0 | 20.0 | — | — |
| Isopropanol | — | — | — | — | — | 20.0 | — | — |
| Dimethicone | 10.0 | — | — | — | — | — | — | — |
| 50 cSt | | | | | | | | |
| Dimethicone | — | 10.0 | — | — | — | — | — | — |
| 500 cSt | | | | | | | | |
| Dimethicone | — | — | 10.0 | 10.0 | 5.0 | 5.0 | 7.0 | 7.0 |
| 200 cSt | | | | | | | | |
| Apricot kernel | 10.0 | 10.0 | 10.0 | 10.0 | 5.0 | 1.0 | 7.0 | 7.0 |
| oil | | | | | | | | |
| Almond oil | — | — | — | — | — | 1.0 | — | — |
| Jojoba oil | — | — | — | — | — | 1.0 | — | — |
| Argo oil | — | — | — | — | — | 1.0 | — | — |
| Olive oil | — | — | — | — | — | 1.0 | — | — |
| Cationic wheat | — | — | — | — | — | — | 1.0 | — |
| hydrolyzate | | | | | | | | |
| Cationic | — | — | — | — | — | — | — | 1.0 |
| keratin | | | | | | | | |
| hydrolyzate | | | | | | | | |
| 3 phases at rest | No | No | No | Yes | Yes | Yes | Yes | Yes |
| Miscibility | Yes | No | Yes | Yes | Yes | Yes | Yes | Yes |
| before use | | | | | | | | |

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A hair treatment agent including, based on its weight,
a. at least 80.0 wt. % aqueous carrier,
b. at least 0.1 to 15.0 wt. % at least one dimethicone and/or dimethiconol with a viscosity of 100 to 350 cSt, measured by the Dow Corning test method CTM 0004, and
c. at least 0.1 to 15.0 wt. % at least one oil,
furthermore characterized in that the composition includes three phases that are visibly separate from one another.

2. The agent according to claim 1, wherein, besides water, the aqueous carrier includes an alcohol selected from the group consisting of methanol, ethanol, propanol, isopropanol, butanol, isobutanol, tert.-butanol, n-pentanol, isopentanols, n-hexanol, isohexanols, glycol, glycerol, 1,2-pentanediol, 1,5-pentanediol, 1,2-hexanediol, 1,6-hexanediol, and mixtures thereof.

3. The agent according claim 1, wherein the ingredients b) and c) are included at a ratio to one another of 5:1 to 1:5.

4. The agent according to claim 1, wherein the oil c) is selected from the group consisting of apricot kernel oil, argan oil, babassu oil, cottonseed oil, bitter almond oil, cashew oil, pomegranate seed oil, grapefruit seed oil, rosehip seed oil, hazelnut oil, raspberry seed oil, elderberry seed oil, blackcurrant seed oil, jojoba oil, cherry kernel oil, coconut oil, bay oil, macadamia nut oil, almond oil, mango butter, passion fruit oil, manila oil, evening primrose oil, olive oil, olive kernel oil, patchouli oil, peach kernel oil, plum kernel oil, pecan nut oil, pistachio oil, sacha inchi oil, sea buckthorn berry oil, sea buckthorn seed oil, sesame oil, shea butter, sweet almond oil, tea tree oil, grape seed oil, walnut oil, wild rose oil, the liquid fractions of coconut oil as well, and mixtures thereof.

5. The agent according to claim 1, wherein the density of the oil is 0.75 to 0.95 g/cm³.

6. The agent according to claim 1, further including a cationic protein hydrolyzate.

7. The agent according to claim 6, wherein the cationic protein hydrolyzate is selected from the group consisting of cationic wheat protein hydrolyzates and cationic keratin hydrolyzates.

8. The agent according to claim 1, further including—based on its weight—0.05 to 7.5 wt. % cationic polymer(s) selected from the group consisting of
poly(methacryloyloxyethyltrimethylammonium chloride) (INCI: polyquarternium-37);
quaternized cellulose derivatives (INCI: polyquarternium 10) and/or
cationic alkyl polyglycosides;
cationized honey;
cationic guar derivatives;
polymeric dimethyldiallylammonium salts and copolymers thereof with esters and amides of acrylic acid and methacrylic acid;
copolymers of vinylpyrrolidone with quaternized derivatives of dialkyl aminoalkyl acrylate and methacrylate;
vinylpyrrolidone-vinylimidazolium methochloride copolymers;
quaternized polyvinyl alcohol;
polyquarternium 2;
polyquarternium-7;
polyquarternium-16;
polyquarternium 17;
polyquarternium 18;
polyquarternium 24; and
polyquarternium 27.

9. A method for human haircare, which includes:
applying an agent according to claim 1 onto the hair and leaving the agent there until the next hair wash.
